(19) United States
(12) Patent Application Publication
(10) Pub. No.: US 2005/0100905 A1
(43) Pub. Date: May 12, 2005

(54) Novel method for production of dna biochips and applications thereof

(75) Inventors: Pierre Nassoy, Suresnes (FR); Marie-Claude Potier, Asnieres (FR); M. Luc Talini, Paris (FR); Nathalie Gibelin, Chatenay Malabry (FR); Jean Rossier, Paris (FR)

Correspondence Address: HELLER EHRMAN WHITE & MCAULIFFE LLP, 1717 RHODE ISLAND AVE, NW, WASHINGTON, DC, 20036-3001, US

(73) Assignees: Centre National de la Recherche Scientifique (CNRS, Paris (FR); Institut Curie, Paris (FR)

(21) Appl. No.: 10/504,163

(22) PCT Filed: Feb. 13, 2003
(86) PCT No.: PCT/FR03/00464

(30) Foreign Application Priority Data
Feb. 13, 2002 (FR) 02 01791

(51) Int. Cl. C12Q 1/68; C12M 1/34; B05D 3/00

(52) U.S. Cl. 435/6; 435/287.2; 427/2.11

(57) ABSTRACT

The invention relates to a method for production of an activated biochip for covalent fixing of oligonucleotide probes to a solid support by means of a spacer compound of the NHS-PEG-VS type and biochips produced by the above method. The invention further relates to methods for detection of nucleic acids in a sample or methods for screening compounds which may be specifically bound to oligonucleotide probes in which said biochips are used. The invention also relates to detection kits for quantitative or qualitative analysis of nucleic acids in a sample comprising said biochips and use of the above as affinity matrix for purification of nucleic acids, for sequencing nucleic acids, for the qualitative or quantitative analysis of the expression of genes or for the study and detection of genetic polymorphism.

The present invention relates to a method for producing an activated biochip for the covalent attachment of oligonucleotide probes to a solid support by means of a spacer compound of the NHS-PEG-VS type, and also to the biochips which can be obtained by such a method. The invention also comprises methods for detecting nucleic acids in a sample or methods for screening compounds capable of specifically binding to oligonucleotides probes, in which the biochips according to the invention are used. The present invention also relates to kits for detecting or for quantitatively or qualitatively analyzing nucleic acids in a sample, comprising such biochips, and to the use of the latter as an affinity matrix for purifying a nucleic acid, for sequencing a nucleic acid, for qualitatively or quantitatively analyzing the expression of genes, or else for studying and detecting genetic polymorphism.

Many techniques or devices for analyzing biological samples have been developed in recent years, in particular for analyzing in parallel large amounts of nucleic acids or proteins, especially subsequent to the development of genomics or of proteomics.

Among these techniques or devices, the supports for carrying out the high throughput analysis of nucleic acids, such as biochips or DNA chips (also called microarrays or macroarrays) have been the subject of many studies.

These biochips can in particular be produced from a functionalized support, that is generally solid, to which given nucleic acids (nucleic acid probes) have been attached by covalent bonding and localized, and to which nucleic acid probes the nucleic acids whose detection or identification in the biological sample is being sought will bind specifically by pairing (or specific hybridization) or by recognition of an affinity site, respectively.

Among the documents which describe the techniques relating to DNA biochips, mention may in particular be made of:
the review article by J. Wang (Nucleic Acids Research, 28, 16, 3011-3016, 2000), which gives an overview summarizing the main known techniques relating to DNA chips, and the document Schubhart et al. (Nucleic Acids Research, 28, 10, e47, 2000) which draws up a list of the problems with which the designers of these chips are confronted;
the patent document granted under No. U.S. Pat. No. 6,030,782, which describes grafting with a mercaptosilanized surface of nucleic acids modified with a sulfhydryl or disulfide group, and the article by Bamdad (Biophysical Journal, 75, 1997-2003, 1998), which describes the production of surfaces exhibiting DNAs by incorporation of composite molecules, DNA-thiols, into self-assembled monolayers (SAMs);
the international patent application published under No. WO 00/43539, which proposes the immobilization of molecules such as oligonucleotides by means of polyfunctional polymers (“polymer brushes”), thereby making it possible to increase the grafting density. These polymers can be obtained from hydroxyethyl methacrylate, acrylamide or vinylpyrrolidone;
the international patent application published under No. WO 00/36145, which, for its part, describes a method for producing DNA chips comprising the polymerization, on a metal layer-type substrate, of a copolymer of pyrrole and of functionalized pyrrole, the binding of a crosslinking agent to the functionalized pyrrole, and then the binding of a biological probe (such as an oligonucleotide). The crosslinking agent may be bifunctional and may, for example, have an N-hydroxysuccinimide ester function and a maleimide function;
the international patent application published under No. WO 98/20020, which also describes the high-density immobilization of nucleic acids on solid supports, this time by bringing a nucleic acid containing a thiol group into contact with a support having a group which reacts with this thiol, optionally by means of a crosslinking agent;
the article by Penchovsky et al. (Nucleic Acids Research, 28, 22, e98, 2000), which describes a method for immobilizing oligonucleotides on aminated latex beads, by means of a crosslinking agent which reacts under the action of light; and
the international patent applications published under Nos. WO 99/16907, WO 00/40593 and WO 00/44939 of the company Surmodics (which produces slides for the depositing of oligonucleotides functionalized with an amine). These applications describe in particular the attachment of nucleic acids to surfaces such as glass, by means of a polymeric backbone to which are attached one or more “photochemically active” groups on one side of the polymer (for the grafting to the surface) and “thermochemically active” groups on the other side (for the grafting with the functionalized nucleic acid).

As regards the use of a heterobifunctional poly(ethylene glycol) (PEG), the international patent application published under the No. WO 95/13312 by the company Shearwater Polymers (which became Nektar), which mentions the therapeutic use of such a spacer agent for grafting therapeutic agents onto proteins, should be noted.

Among the biochips already produced or in the process of being produced, those which can be coated with both single-stranded short-sequence (a few tens of bases) nucleic acid probes, which can be synthesized chemically, and much longer double-stranded sequences, such as those derived from PCR products, ranging from 200 to a few thousand base pairs, are the subject of great interest.

In fact, for short-sequence oligonucleotides, supports covered with polylysine, for example, cannot be used, since the nucleic acids are adsorbed on the polylysine in a flat manner, and access to the sequence during hybridization is difficult or even impossible if the deposited strand is short. It is therefore necessary to be able to graft the short oligonucleotide via one of its ends in order to free the access to the sequence during the subsequent hybridization with the target nucleic acid.

A few commercial supports exist which enable this grafting by covalent bonding. In general, the results obtained with these biochips are not completely satisfactory:
the background noise is too great;
the hybridization signal is too weak;
there are smears (streaks of fluorescence around the spots after hybridization);
the surface wetting properties are not satisfactory (the deposits spread out until one spot overlaps another, heterogeneities in the form of rings appear), which does not make it possible to act on the grafting density or to obtain spots of correct size (of average diameter ranging from 50 to 200 μm during deposition);
there is no spacer arm to the give the grafted probe mobility.

For this, it would be desirable to be able also to have a biochip in which the support makes it possible, after attachment, not to affect the tertiary structure (three-dimensional conformation) and to give sufficient mobility for it to be possible for specific hybridization between single-stranded nucleic acids to take place.

Finally, it would be desirable to be able to also have a biochip for which the protocol for carrying out the immobilization of the oligonucleotide probes on a support, such as glass, for the purpose of producing these biochips is a protocol that is simple (minimum of steps, if possible of “light” chemistry), rapid (this point is all the more important since the volumes used on each “spot” are very small and evaporate rapidly, it is therefore essential for the covalent grafting to be rapid and for it to be possible for the excess probes to be easily removed from the surface (without leaving streaks)) and reproducible.

It appears, with regard to the biochips tested and described in the documents already published, that none of these biochips corresponds to these criteria.

Thus, there is still the need for a biochip having these characteristics.

This is precisely the subject of the present invention.

The inventors have surprisingly demonstrated that the use of a heterobifunctional spacer compound NHS-PEG-VS of formula (I) below, attached by covalent bonding to a prefunctionalized solid support, makes it possible to obtain biochips corresponding to this expectation.

Thus, a subject of the present invention is a method for producing an activated biochip for the covalent attachment of oligonucleotide probes, said biochip comprising a solid support prefunctionalized with a thiol or amine function, characterized in that it comprises a step of covalent attachment, under appropriate conditions, to said functionalized support of a spacer compound NHS-PEG-VS of formula (I): 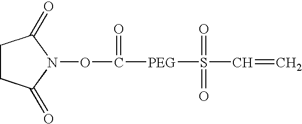
in which PEG denotes poly(ethylene glycol) of formula
HO—(CH₂CH₂O)ₙCH₂CH₂—OH,
where n is an integer chosen such that the molecular mass of the compound NHS-PEG-VS of formula (I) is between 500 and 5000, preferably between 2000 and 4000, more preferably in the region of 3400.

The term “activated biochip” is intended to denote, in the present description, a solid support as defined below, to which will be attached, by covalent bonding, the spacer compounds of formula (I) capable of interacting with the nucleic acid probes, but not yet coated with these said probes.

The terms “nucleic acid”, “nucleic acid probe”, “nucleic acid sequence”, “polynucleotide”, “oligonucleotide”, “polynucleotide sequence” and “nucleotide sequence”, which terms will be employed without distinction in the present description, are intended to denote a precise series of modified or unmodified nucleotides making it possible to define a fragment or a region of a nucleic acid, which may or may not comprise unnatural nucleotides, and which may correspond both to a double-stranded DNA, a single-stranded DNA, a PNA (for peptide nucleic acid) or an LNA (for locked nucleic acid), and to products of transcription of said DNAs, such as RNA.

The term “oligonucleotide probe” or “nucleic acid probe” will be intended to denote here the functionalized oligonucleotide which will be deposited (or spotted) and attached by covalent bonding to said spacer compound on the functionalized solid support, as opposed to the target nucleic acid derived from the biological sample whose detection and identification is being sought.

In a preferred embodiment, the method for producing an activated biochip according to the invention is characterized in that said solid support is chosen from solid supports made of glass, of plastic, of Nylon®, of Kevlar®, of silicone, of silicon, or else of polysaccharides or poly(heterosaccharides), such as cellulose, and preferably made of glass.

This support may be of any shape (flat slide, microbeads, etc.).

In a particularly preferred embodiment, the method for producing an activated biochip according to the invention is characterized in that said solit support made of glass is functionalized by silanization.

In an embodiment that is also preferred, the method for producing an activated biochip according to the invention is characterized in that said solid support is functionalized with an amine function when said oligonucleotide probes intended to be attached are functionalized with an end thiol function.

When said solid support, in particular made of glass, is functionalized with an amine function, this functionalization is preferably carried out in the presence of an aminosilane, preferably N-(2-aminoethyl)-3-aminopropyltrimethoxysilane.

In an embodiment that is also preferred, the method for producing an activated biochip according to the invention is characterized in that said solid support is functionalized with a thiol function when said oligonucleotide probes intended to be attached are functionalized with an end amine function.

When said solid support, in particular made of glass, is functionalized with a thiol function, this functionalization is preferably carried out in the presence of mercaptosilane, preferably (3-mercaptopropyl)trimethoxysilane.

A subject of the present invention is also a method for producing a deactivated biochip for the covalent attachment of oligonucleotide probes functionalized with an end amine function, characterized in that it comprises the following steps:
a) activation of the biochip by means of a method for producing an activated biochip for the covalent attachment of oligonucleotide probes functionalized with an end amine function; and
b) hydrolysis, in the presence of an aqueous solution, preferably of pure water, of the free NHS functions of the spacer compounds NHS-PEG-VS of formula (I) attached to the solid support.

A subject of the present invention is also a method for producing a regenerated biochip for the covalent attachment of oligonucleotide probes functionalized with an end amine function, characterized in that it comprises the following steps:
A) deactivation of the biochip by means of the above method for producing a deactivated biochip according to the invention;
B) regeneration, in the presence of 1-ethyl-3[3-(dimethylamino)propyl]carbodiimide hydrochloride (EDC) and of NHS, of the carboxylate groups obtained at the free end of said spacer compounds after hydrolysis of the free NHS functions initially present, and, where appropriate,
C) at least one step consisting in washing the biochip obtained in step B), preferably in deionized water.

In a preferred embodiment, the method for producing an activated, deactivated or regenerated biochip according to the invention is characterized in that it comprises a step in which the biochip obtained is frozen, dried or lyophilized, preferably dried under an inert atmosphere, for instance under nitrogen, and protected against moisture.

In another aspect, a subject of the present invention is a method for producing a biochip coated with oligonucleotide probes, characterized in that it comprises the following steps:
α) preparation of an activated or regenerated biochip by means of a method according to the invention;
β) depositing and attachment, by covalent bonding, under the appropriate conditions:
   either of said oligonucleotide probes prefunctionalized with a thiol function, if said solid support has been functionalized with an amine function,
   or of said oligonucleotide probes prefunctionalized with an amine function, if said solid support has been functionalized with a thiol function; and, where appropriate,
γ) removal of the oligonucleotide probes which have not attached to the support, by means of at least one step consisting in rinsing the support under appropriate conditions, preferably in deionized water.

The present invention also relates to a method for producing a biochip comprising a solid support prefunctionalized with a thiol function, and then activated by spotting of NHS-PEG-VS of formula (I) and coated with oligonucleotide probes according to the invention, characterized in that it also comprises the following step:
δ) deactivation, in the presence of amino compounds under the appropriate conditions, of the NHS functions of the spacer compound which have not interacted with the amine functions of the oligonucleotide probes.

In a preferred embodiment, said compound for deactivating the NHS functions of the spacer compound in step δ) is chosen from amino compounds having a primary amine, preferably ethanolamine or methoxy-PEG-NH₂, in particular as available, for the latter, from the company Shearwater Polymers (USA).

The present invention also relates to a method for producing a biochip comprising a solid support prefunctionalized with an amine function, and then activated by spotting of NHS-PEG-VS of formula (I) and coated with oligonucleotide probes, characterized in that it also comprises the following step:
δ) reduction, under the appropriate conditions, of the surface charges in the presence of anionic compounds or compounds capable of establishing covalent bonds with the amine groups and of producing a species that is neutral or negative under the appropriate conditions, preferably in the presence of methyl N-succinimidyl adipate (MSA).

The present invention also relates to a method for producing a biochip coated with oligonucleotide probes according to the invention, characterized in that it comprises a step in which the biochip obtained is conserved in a dry place, in the dark and/or in an inert atmosphere.

Preferably, said oligonucleotide probes are single-stranded DNAs or RNAs, preferably DNAs or RNAs the size of which is between 15 and 7000 bp, preferably between 20 and 1000 bp, between 20 and 500 bp, between 20 and 250 bp, between 20 and 100 bp, between 20 and 80 bp or between 35 and 80 bp.

These probe DNAs or RNAs can be obtained by chemical synthesis, or from genomic DNA, from RNA or from mRNA, or from fragments thereof, extracted from cells, in particular for cDNAs after reverse transcription of these RNAs, or else in the form of a PCR fragment obtained by RT-PCR from these RNAs, or by PCR from these genomic DNAs (“RT-PCR” for method referred to as reverse transcription polymerized chain reaction).

More preferably, the methods for producing a biochip coated with oligonucleotide probes according to the invention are characterized in that said oligonucleotide probes are deposited in the form of spots the average diameter of which is between 20 μm and 500 μm, preferably between 50 μm and 200 μm, and, where appropriate, in that the average distance between the center of each of the oligonucleotide probe spots is between 80 μm and 400 μm.

More preferably, the methods for producing a biochip coated with oligonucleotide probes according to the invention are characterized in that the number of said oligonucleotide probe spots is between 2 and 10⁵, preferably between 2 and 10⁴, 2 and 10³, 2 and 4×10², 2 and 10², even more preferably between 50 and 10³ and between 50 and 4×10² per cm².

In a novel aspect, a subject of the present invention is a biochip comprising a solid support prefunctionalized with a thiol or amine function, characterized in that it comprises a spacer compound NHS-PEG-VS of formula (I): 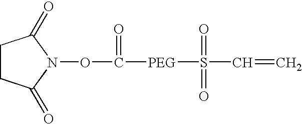
in which PEG denotes poly(ethylene glycol) of formula
HO—(CH₂CH₂O)ₙCH₂CH₂—OH,
where n is an integer chosen such that the molecular mass of the compound NHS-PEG-VS of formula (I) is between 500 and 5000, preferably between 200 and 4000, and in the region of 3400,
said spacer compound being attached to said solid support via a covalent bond resulting either from interaction between the thiol function of said functionalized support and the vinylsulfone function of the spacer compound of formula (I), or from interaction between the amine function of said functionalized support and the NHS function of the spacer compound of formula (I).

Preferably, the biochip according to the invention is characterized in that it also comprises at least one oligonucleotide probe, prefunctionalized with a thiol or amine function, attached to said solid support via a covalent bond resulting either from interaction between the free NHS function of said spacer compound of formula (I) and an amine function of said oligonucleotide probe, or from interaction between the free vinylsulfone function of the spacer compound of formula (I) and a thiol function of said oligonucleotide probe.

Also preferably, the biochip according to the invention is characterized in that said attached oligonucleotide probes are single-stranded DNAs or RNAs, preferably DNAs or RNAs the size of which is between 15 and 7000 bp, preferably between 20 and 1000 bp, between 20 and 500 bp, between 20 and 250 bp, between 20 and 100 bp, between 20 and 80 bp, or between 35 and 80 bp.

Also preferably, the biochip according to the invention is characterized in that said oligonucleotide probes are deposited in the form of spots the diameter of which is between 20 μm and 500 μm, preferably between 50 μm and 200 μm, and, where appropriate, in that the average distance between the center of each of the oligonucleotide probe spots is between 80 μm and 400 μm.

Also preferably, the biochip according to the invention is characterized in that the number of said oligonucleotide probe spots is between 2 and 10⁵, preferably between 2 and 10⁴, 2 and 10³, 2 and 4×10², 2 and 10², even more preferably between 50 and 10³, and between 50 and 4×10² per cm².

Also preferably, the biochip according to the invention is characterized in that said solid support is chosen from solid supports made of glass, of plastic, of Nylon®, of Kevlar®, of silicone, of silicon, of polysaccharides or poly(heterosaccharides), and preferably made of glass, preferably silanized.

A subject of the present invention is also an activated, deactivated or regenerated biochip or else a biochip coated with oligonucleotide probes, which can be obtained by means of a method according to the invention.

According to yet another aspect, the present invention comprises the use of a biochip according to the invention, for detecting nucleic acids in a sample.

According to yet another aspect, the present invention relates to a kit or set for detecting or qualitatively or quantitatively analyzing nucleic acids in a sample, characterized in that it comprises a biochip according to the invention.

A subject of the present invention is also a method for detecting nucleic acids in a sample, characterized in that it comprises the following steps:
a) depositing the sample containing the target nucleic acids, the detection of whose presence is being sought, onto a biochip coated with oligonucleotide probes according to the invention, under conditions which allow the specific hybridization of these target nucleic acids with said oligonucleotide probes;
b) where appropriate, rinsing the biochip obtained in step a) under the appropriate conditions in order to remove the nucleic acids of the sample which have not been captured by hybridization; and
c) detecting the nucleic acids captured on the biochip by hybridization.

The expression “conditions which allow the specific hybridization of target nucleic acids with said oligonucleotide probes” preferably involves high stringency conditions, in particular as defined below or as mentioned, without being limited thereto, in the examples below.

A hybridization under high stringency conditions means that the conditions of temperature and of ionic strength are chosen such that they enable the hybridization between two complementary fragments of DNA or of RNA/DNA to be maintained. By way of illustration, high stringency conditions in the hybridization step for the purpose of defining the hybridization conditions described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10× Denhardt's, 5% of dextran sulfate and 1% of salmon sperm DNA; (2) actual hybridization for 20 hours at a temperature which depends on the length of the probe (i.e.: 42° C., for a probe >100 nucleotides in length) followed by 2 washes of 20 minutes at 20° C. in 2×SSC+2% SDS, 1 wash of 20 minutes at 20° C. in 0.1×SSC+0.1% SDS. The final wash is carried out in 0.1×SSC+0.1% SDS for 30 minutes at 60° C. for a probe >100 nucleotides in length. The high stringency hybridization conditions described above for a polynucleotide of defined length can be adapted by those skilled in the art for longer or shorter oligonucleotides, according to the teaching of Sambrook et al. (1989, Molecular cloning: a laboratory manual, 2^{nd} Ed. Cold Spring Harbor).

The invention also comprises a method for detecting nucleic acids in a sample according to the invention, characterized in that the nucleic acids, the detection of whose presence is being sought, are prelabeled at one of their ends with a label capable of directly or indirectly generating a detectable signal, preferably a signal detectable by fluorescence.

The invention also comprises a method for detecting nucleic acids according to the present invention, characterized in that at least two of the nucleic acids, the detection of whose presence is being sought, are prelabeled with a different label.

Preferably, said labels are chosen from cyanin derivatives, preferably chosen from sulfonated derivatives of cyanin, in particular the compounds Cy5 or Cy3, nanocrystals (Migyong Han et al, Nature Biotechnology, 19, 631-635, 2001) or nanoparticles (company Genicon Science).

A subject of the present invention is also the use of a biochip according to the invention, as an affinity matrix or for purifying a nucleic acid.

A subject of the present invention is also the use of a biochip according to the invention, for sequencing a nucleic acid, for qualitatively or quantitatively analyzing the expression of genes, or else for studying and detecting genetic polymorphisms (also called SNPs for “single nucleotide polymorphisms” or SNIPs).

For example, but without being limited thereto, DNA probes corresponding to known genes are deposited onto the biochips according to the invention. Each deposit or spot may contain several thousand oligonucleotide probes corresponding to the same gene, and from one spot to the other, the oligonucleotide probes will correspond to another gene, or to a gene having a different polymorphism.

The genomic DNAs, or messenger RNAs, or fragments thereof, of the tissue or of the cell intended to be studied may be extracted and labeled with fluorochromes (the DNAs or mRNA may in particular be converted into complementary DNAs (cDNAs) by reverse transcription and, where appropriate, multiplied by PCR or RT-PCR techniques).

These DNAs, cDNAs or RNAs will then be deposited onto the biochip coated with oligonucleotide probes and, where appropriate, will bind by specific hybridization with the pre-deposited oligonucleotide probes which correspond to them. The amount of signal, in particular of fluorescence, thus corresponding to the amount of target nucleic acids hybridized, which will in particular be proportional to the initial amount of mRNAs extracted, if the target DNAs deposited are cDNAs complementary to transcribed mRNAs, will then be detected on each spot. The transcriptional activity of the cell may thus be measured for certain genes.

There are consequently numerous applications for these DNA biochips, such as transcriptional studies, diagnosis (the search for a mutation), the search for therapeutic targets, genetic mapping of individuals.

For the general applications of these biochips, reference may in particular be made to the numerous documents already published on this subject, in which documents the methods implemented for these applications using activated or regenerated biochips, such as those of the present invention, are completely explained.

In yet another aspect, the invention relates to a method for screening compounds or cells capable of specifically binding to a given oligonucleotide, characterized in that it comprises the following steps:
a) bringing said test compound into contact with a biochip according to the invention, under the conditions for the possible specific binding of said test compound or of said test cell with said given oligonucleotide, said biochip comprising at least one spot of oligonucleotide probes containing said given oligonucleotides attached to its solid support and, where appropriate, said compound or said cell being labeled with a label capable of directly or indirectly generating a detectable signal;
b) removing, by means of at least one washing step under the appropriate conditions, the test compounds or cells not specifically bound to said given oligonucleotide; and
c) selecting the compound or said cell specifically bound to said given oligonucleotide, where appropriate by selecting the compound or said cell whose signal has been detected at the spot containing said given oligonucleotide.

Finally, in a last aspect, a subject of the present invention is a diagnostic or investigating instrument or device comprising a biochip according to the invention.

Other characteristics and advantages of the invention emerge in the remainder of the description with the examples and the figures for which the legends are represented below.

### FIGURE LEGENDS

FIGS. 1A to 1D: Grafting of oligonucleotides functionalized with an amino group (NH₂-terminated)

FIG. 1A: glass surface (silanols Si—OH).

FIG. 1B: glass surface functionalized with mercaptosilanes (SH-terminated).

FIG. 1C: grafting of heterobifunctional PEG (NHS-PEG-VS). The NHS ends remain free and reactive.

FIG. 1D: attachment of oligonucleotides functionalized with an amino group (NH₂-terminated).

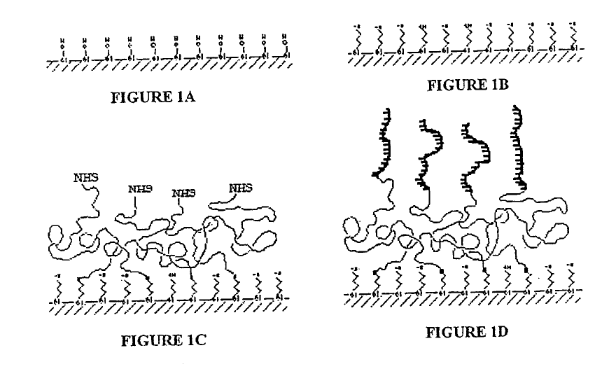

### EXAMPLE 1

### Support for Oligonucleotide Probes Functionalized with an Amine Group: Method 1

1. Principle

Use of a heterobifunctional PEG carrying NHS and VS functions.

The surface is initially silanized (functionalization) in such a way as to obtain SH functions (surface thiol). The VS function of the heterobifunctional PEG which is then deposited (activation) will react with the thiols of the surface to form a covalent bond. The oligonucleotides carrying an amine function at one of their ends are finally spotted. This amine function will react with the NHS function of the heterobifunctional PEG to form a covalent bond.

2. Experimental Protocol

a) Silanization

GOLD SEAL glass slides are used. These slides are cleaned with 300 ml of a sulfuric acid/hydrogen peroxide (70/30, v/v) mixture for 15 minutes (Piranha mixture).

The slides are rinsed with pure water and then with methanol.

The silanization bath is made up of:
60 ml of pure methanol for synthesis (SDS ref.: 0930221);
510 μl of acetic acid;
2.55 ml of pure water; and
1.275 ml of (3-mercaptopropyl)trimethoxysilane (SIGMA, EE No. 224-588-5).

The slides are immersed in this bath for 2 hours.

The slides are then rinsed with pure methanol and then argon-dried, and finally placed in an incubator at 94° C. for 15 minutes.

The slides are stored under vaccum in a dessicator.

b) Application of the Heterobifunctional PEG

Preparation of a solution of heterobifunctional PEG in 1×PBS buffer (phosphate buffered saline) at pH 7. This pH value is optimal for the reaction between the thiols of the surface and the VS function of the PEG.

For one slide:
2 mg of NHS-PEG-VS, MW 3400 (Shearwater Polymers); and
2 ml of 1×PBS.

The solution is deposited on the surface of the slide to be treated for 45 minutes.

The slides are then rinsed with deionized water and argon-dried.

### EXAMPLE 2

### Support for Oligonucleotide Probes Functionalized with a Thiol Group: Method 2

1. Principle

It is the same principle as for method 1, except that, here, the surface of the slides is silanized with amine functions. Thus, the NHS function of the PEG will react with these surface groups and the remaining VS group may react with oligonucleotides having an end thiol function.

2. Experimental Protocol

a) Silanization

The slides are, as for the experimental protocol in example 1, washed with the Piranha mixture.

The composition of the silanization bath changes:
60 ml of pure methanol for synthesis (SDS ref.: 0930221);
510 μl of acetic acid;
2.55 ml of pure water;
1.275 ml of silane: N-[3-(trimethoxysilyl)-propyl]diethylenetriamine, 97% (ALDRICH 10, 488-4).

The solution is deposited onto the surface of the slide to be treated for 45 minutes.

The slides are then rinsed with deionized water and argon-dried.

b) Application of the Heterobifunctional PEG

Composition of the solution (for one slide):
2 mg of NHS-PEG-VS MW 3400, Shearwater Polymers;
2 ml of carbonate-bicarbonate (CB) buffer, pH 8.4.

The pH of 8.4 is optimal for the chemical reaction between the NHS function of the PEG and the NH₂ functions of the silanized surface.

The solution is deposited onto the surface of the slide to be treated for 45 minutes.

The slides are then rinsed with deionized water and argon-dried.

### EXAMPLE 3

### Deactivated then Regenerated Support for Oligonucleotide or Peptide Probes Functionalized with an Amine Group

1. Principle

The NHS function is sensitive to moisture and becomes deactivated in a few days. To remedy this, a method was developed which consists in deactivating the function in a stable form and then in regenerating it at the time the oligonucleotide probes are spotted.

2. Experimental Protocol

a) Deactivation

The slides are first prepared as for Method 1. The NHS function is then hydrolyzed by immersing the slides in a bath of pure water until the time of deactivation. The NHS function converts to a carboxylate (COO⁻).

b) Regeneration

The protocol of Patel et al. (Langmuir, 13:06485-6490, 1997) is applied here.

Briefly:

A solution made up of the following is prepared in pure water:
15 mM of NHS (Fluka), preferably dissolved beforehand in DMSO (it is also possible to use sulfo-NHS instead of NHS, which is soluble in water); and
5 mM of EDC.

The slides are then immersed in the solution for 10 minutes.

The slides are then rinsed with pure water and are argon-dried.

### EXAMPLE 4

### Capping of the Regions of the Support Exhibiting Active NHS Groups of the Spacer Compound Which have not Interacted with the Probes for Method 1

1. Principle

After the spotting of the oligonucleotides, the regions around these spots still have active groups of NHS or VS type depending on the method used. This is in particular a hindrance for Method 1 as described in example 1 for which the NHS active group is reactive with amine groups. Now, these amine groups are found in certain nucleotide bases. These amine groups are, in particular for the bases, much less reactive than the primary amine groups which serve as attachment functions for the oligonucleotides. Thus, during the spotting, the amines of the bases do not compete (or compete very little) with the terminal amines of the functionalized oligonucleotide probes. On the other hand, during hybridization, the target nucleic acids, such as cDNAs, which are deposited onto the support have only the amines constituting their bases. There is therefore a risk of a reaction between the amines of the bases of the target nucleic acids and the NHSs remaining around the oligonucleotide probe spots, the consequence of which is an increase in the background noise. It is therefore preferable to deactivate these NHS sites that are still active.

2. Protocol for Capping the Supports of Method 1 as Described in Example 1

2.1 Protocol 1 Using Ethanolamine as Agent for Deactivating the NHS Groups Which have not Interacted with the Probes.

Capping Solution:

40 ml of phosphate buffer (HPO₄/H₂PO₄), pH=8.2, 150 mM;
1 ml of ethanolamine;
2 ml of 10% SDS; and
the volume is made up to 200 ml with milliQ water.
   Washing Solution:
100 ml of 20×SSC;
5 ml of 10% SDS; and
the volume is made up to 500 ml with milliQ water.

The slides are placed in the blocking solution at 50° C. for 15 min:
rinsing with milliQ water for 4 min;
washing of the slides with the washing solution for 15 min;
rinsing with milliQ water for 6 min; and
centrifugation at 800 rpm (50 g) for 3 min.
   2.2 Protocol 2 Using a Monofunctional PEG as Agent for Deactivating the NHS Groups Which have not Interacted with the Probes.

For this second protocol, a monofunctional PEG having an amine group at one of its ends is used. This amino group reacts with the NHSs by forming a covalent bond. The surface around the spots on which the probes have been spotted is then covered with PEG.

A solution is prepared, containing:
2 ml of 150 mM phosphate buffer (HPO₄/H₂PO₄) at pH 8.2; and
2 mg of methoxy-PEG-NH₂ (Shearwater Polymers).

The slide is immersed in this solution for 45 minutes and is then rinsed with pure water and argon-dried.

### EXAMPLE 5

### Spotting of the Regions of the Support Exhibiting Active VS Groups of the Spacer Compound Which have not Interacted with the Probes for Method 2

Since there is no thiol function in the bases constituting nucleic acids, such as DNA, it is not therefore necessary to deactivate the free VS sites for the spotting of oligonucleotide probes. Only rinsing with pure water is carried out in order to remove, in this case, the oligonucleotides which have not become grafted.

In general, any thiolated compounds, and for example N-ethylmaleimide, iodoacetate derivatives (sodium tetrathionate, Ellman's reagent), aziridines or acryloyl derivatives can be used.

However, in order to isolate or to reduce the surface charges in Method 2, it is preferable to deposit, before spotting the probes, under the appropriate conditions, anionic compounds or compounds capable of establishing covalent bonds with the amine groups and of producing a species that is neutral or negative under the appropriate conditions, such as, for example, according to the protocol herein after using methyl N-succinimidyl adipate (MSA).

Protocol using MSA as agent for neutralizing the surface charges in Method 2.

A solution of MSA in 1×PBS, pH 7.4 (1 mg of MSA per ml of PBS) is deposited onto the surface. The surface is covered with a plastic slide. The MSA solution is left in contact with the surface for 1 hour. The surface is then rinsed with pure water and nitrogen-dried.

### EXAMPLE 6

### Experiments Consisting in Hybridizing the Slides with a Solution of Oligonucleotide Probes: Influence of pH on the Oligonucleotide Grafting

1. Principle of the Experiments

Oligonucleotide probes of 50 bases corresponding to mouse gene fragments were deposited onto functionalized glass slides to which the spacer compound NHS-PEG-VS had been previously attached. Three nucleotide sequences were used:

| | |
|---|---|
| GTGCCTCACGGTGGTTGCCATCACTGTCTTCATGTTCGAGTATTTCAGCC; | (SEQ ID No. 1) |
| TTTTGAGATOTGGCTTCATTTCGACGCTGACGGAACTGGTTACCTGGAAG; | (SEQ ID No. 2) |
| and | |
| AACGCCCATCTTAAAATCGACGCCTGTCTCTCCCCCATTGCTCTTACCAG. | (SEQ ID No. 3) |

For each probe sequence, three types of oligonucleotides were deposited:
NH₂-functionalized in the 3′ position;
SH-functionalized in the 3′ position; and
nonfunctionalized.

The slides thus obtained were compared to the slides of various companies.

2. Material

The oligonucleotides are deposited using a “spotter” from the company GeneMachines (OmniGrid). This spot is a pin spotter (Majer Precision) which makes it possible to make spots of a few ml. Between each spot, the pin is washed in order to prevent contaminations from one spot to another.

The slides are then read, after hybridization, using a scanner (ScanArray 3000 GSI) and the images are analyzed using the Imagene 4.1 software (Biodiscovery).

3. Experiments Consisting in Hybridizing Oligonucleotides on the Slides for Aminated Oligonucleotides

a) Preparation of the Slides

The slides according to the present invention are prepared according to Method 1 and Method 2. The results obtained with these slides were compared to those obtained under the same conditions for use with slides sold by the company SurModics (SurModics Inc., Eden Prairie, Minn.) under the reference “3D-LINK™ activated slides” (batch DN01B058 packet No. 19), which are activated slides capable of attaching NH₂-functionalized nucleotide probes having a free amine function (“amine-binding slides”). The slides of the company SurModics are known to be those of the commercial slides currently available that give the highest performance levels. For comparison of chemical methods for covalently immobilizing oligonucleotides on glass slides, reference may, for example, be made to the article by Lindroos et al. (Nucleic Acids Research, 29, 13, e69, 2001), which compares 8 different methods, including a certain number of commercialized slides.

Three oligonucleotide probe sequences and, for each of these probe sequences, three types of modified oligonucleotides (NH₂-functionalized, SH-functionalized, nonfunctionalized) were deposited, each oligonucleotide solution for various pH values (6.8, 7.7 and 8.3). Each sample is spotted twice. Thus, on each slide, 3×3×3×2=54 oligonucleotide spots are deposited.

This matrix of spots was deposited twice on the slide.

In addition, between each oligonucleotide, a double deposit of buffer is carried out in order to eliminate any risk of contamination.

b) Preparation of the Oligonucleotides

The oligonucleotides are resuspended in pure water (milliQ) at a concentration of 0.5 mM.

The concentration of the oligonucleotide solutions is adjusted to 5 μM for the pH values 6,8, 7.7 and 8.3, in 150 mM phosphate buffer, in a total volume of 12 μl.

c) Preparation of the Oligonucleotide Hybridization Solution

The biochips thus coated with oligonucleotide probes are hybridized with a solution containing target oligonucleotides having sequences complementary to the oligonucleotide probes deposited.

Three target oligonucleotides having sequences complementary to the oligonucleotide probes thus spotted were mixed:

| | |
|---|---|
| GGCTGAAATACTCGAACATGAAGACAGTGATGGCAACCACCGTGAGGCAC; | (SEQ ID No. 4) |
| CTTCCAGGTAACCACTTCCGTCAGCGTCGAAATGAAGCCAGATCTCAAAA; | (SEQ ID No. 5) |
| and | |
| CTGGTAAGAGCAATGGGGGAGAGACAGGCGTCGATTTTAAGATGGGCGTT. | (SEQ ID No. 6) |

These target oligonucleotides carry a fluorochrome (Cy5) in the 5′ position.

These complementary target oligonucleotides are firstly suspended in pure water at a concentration of 50 μM.

The following mixture is then prepared:
497 μl of pure water; and
1 μl of each complementary target oligonucleotide (i.e. 3 μl in total).

A mixture of the three target oligonucleotides carrying a fluorochrome (Cy5) in the 5′ position, at a concentration of 0.1 μM for each of these target oligonucleotides, is obtained.

The hybridization solution is then prepared:
1 μl of the solution of complementary target oligonucleotides at 0.1 μM for each;
15.8 μl of pure water;
3.6 μl of 20×SSC (pH 7); and
0.6 μl of 10% SDS,
   i.e. a solution at a concentration of 5 nM per complementary target oligonucleotide.
   d) Hybridization

The biochips coated with oligonucleotide probes are hybridized with the complementary target oligonucleotide solution described above.

12 μl of target oligonucleotide solution are deposited onto each biochip. Each biochip is covered with a plastic cover slip and the biochips are placed in a hybridization chamber (GeneMachines).

The chamber is kept in a water bath overnight at 60° C.

e) Rinsing

Solution 1:
50 ml 20×SSC;
5 ml 10% SDS; and
qs 500 ml of milliQ water.

Solution 2:
5 ml 20×SSC; and
qs 500 ml of milliQ water.

Solution 3:
2.5 ml 20×SSC; and
qs 500 ml of milliQ water.

After hybridization, the slides are rinsed as below.

The slides are first placed in a 4×SSC solution in order to cause the cover slip to drop off, followed by:
rinsing for 2×5 min in solution 1;
rinsing for 1 min in solution 2; and
rinsing for 1 min in solution 3.
   4. Results of the Hybridization Experiments
   4.1 Setting the Scanner

The scanner used has two settings for the reading:
the excitation (LASER) intensity setting; and
the photomultiplier (PMT, used to measure fluorescence intensity) power setting.

The intensity of these two settings is expressed in an arbitraty unit. At the maximum, the two values are at 100.

For some slides, these maximum values saturate the fluorescent signal; it is then necessary to decrease the intensities of the LASER and/or of the PMT.

4.2 Slides for Oligonucleotide Probes Functionalized with an Amino Group: Method 1

For a LASER/PMT setting at 100/100, the signal obtained is saturated for all the oligonucleotide probe spots deposited (signal greater than 65 000). These settings were thus decreased to 75/68.

The results of the experiments are given in Table 1 below.

TABLE 1

| | | | | | Standard |
|---|---|---|---|---|---|
| | | Background | | Mean | deviation |
| | Mean | noise | | diameter | of the |
| | fluorescence | around the | Signal/noise | (μm) | diameters |
| | intensity | spot | ratio | n = 6 | n = 6 |
| oligo NH₂ | 18900 +/− 6550 | 6.4 +/− 0.5 | 3000 +/− 1000 | 96 | 10 |
| pH = 6.8 | | | | | |
| oligo NH₂ | 22400 +/− 5700 | 7 +/− 0.45 | 3200 +/− 800 | 105 | 4 |
| pH = 7.7 | | | | | |
| oligo NH₂ | 34500 +/− 1600 | 6.7 +/− 0.6 | 5200 +/− 450 | 103 | 3 |
| pH = 8.3 | | | | | |
| oligo non- | 4300 +/− 1500 | 5.6 +/− 0.2 | 800 +/− 250 | 93 | 7 |
| functionalized | | | | | |
| pH = 6.8 | | | | | |
| oligo non- | 6500 +/− 1900 | 6.0 +/− 0.4 | 1000 +/− 1100 | 93 | 7.5 |
| functionalized | | | | | |
| pH = 7.7 | | | | | |
| oligo non- | 8000 +/− 2600 | 6.0 +/− 0.2 | 1300 +/− 400 | 101 | 8.5 |
| functionalized | | | | | |
| pH = 8.3 | | | | | |

Legend of Table 1: Experiment consisting of hybridization on a biochip prepared according to Method 1: pH test.

NH₂-Functionalized (“oligo NH₂”) and nonfunctionalized oligonucleotide probes were deposited at various pH values. The chip was hybridized with a mixture of fluorescence-labeled target oligonucleotides complementary to those deposited. 2 matrices of 54 spots were deposited.

The ratios of the fluorescence intensities obtained for the functionalized and nonfunctionalized oligonucleotide probes revealed the very good selectivity of the support for the functionalized oligonucleotide probes which graft by covalent bonding.

The signal to noise ratio is excellent (5000), since the background noise is very low.

The size of the spots is very good, with a very low dispersion of the diameters (low wettability), especially for the oligonucleotide probes functionalized with amino groups.

Finally, the grafting reaction is particularly efficient at basic pH (8.3).

4.3 Slides of Oligonucleotide Probes Functionalized with a Thiol Group: Method 2

The scanner was set on 100/100. The results of the experiment are given in Table 2 below.

TABLE 2

| | | | | | Standard |
|---|---|---|---|---|---|
| | | | | | deviation |
| | Mean | Background | | | of the |
| | fluorescence | noise around | Signal/noise | Mean diameter | diameters |
| | intensity | the spot | ratio | (μm) n = 6 | n = 6 |
| oligo SH | 60600 | 500 | 120 | 155 | 5.5 |
| pH = 6.8 | | | | | |
| oligo SH | 62700 | 525 | 120 | 157 | 8 |
| pH = 7.7 | | | | | |
| oligo SH | 61300 | 463 | 130 | 157 | 10 |
| pH = 8.3 | | | | | |
| oligo non- | 36500 | 390 | 94 | 160 | 11 |
| functionalized | | | | | |
| pH = 6.8 | | | | | |
| oligo non- | 44000 | 470 | 94 | 156 | 10 |
| functionalized | | | | | |
| pH = 7.7 | | | | | |
| oligo non- | 50500 | 425 | 120 | 160 | 10 |
| functionalized | | | | | |
| pH = 8.3 | | | | | |

Legend of Table 2: Experiment consisting of hybridization on a chip prepared according to Method 2: pH test

Oligonucleotide probes functionalized with an SH group (“Oligo SH”) and nonfunctionalized oligonucleotide probes (“Oligo nonfunctionalized”) were deposited in solution at various pH values. The chip was hybridized with a mixture of the fluorescence-labeled target oligonucleotides complementary to the probes deposited.

The oligonucleotide probes functionalized with an SH group attach better than the nonfunctionalized oligonucleotide probes, but the selectivity of the support is weaker than for the slides obtained with Method 1. The pH has fairly little influence on the grafting.

The signal to noise ratio is quite good.

4.4 Slides Obtained from the Supplier Surmodics for Oligonucleotide Probes Functionalized with an Amino Group

The scanner setting is 100/100.

Results: see Table 3 below.

TABLE 3

| | | Background | | | Standard |
|---|---|---|---|---|---|
| | Mean | noise | | Mean | deviation |
| | fluorescence | around the | Signal/noise | diameter | of the |
| | intensity | spot | ratio | (μm) | diameters |
| oligo NH₂ | 19000 +/− 13500 | 60 +/− 10 | 310 +/− 175 | 133 | 10 |
| pH = 6.8 | | | | | |
| oligo NH₂ | 44000 +/− 10500 | 65 +/− 10 | 700 +/− 200 | 127 | 10 |
| pH = 7.7 | | | | | |
| oligo NH₂ | 50000 +/− 11600 | 70 +/− 10 | 700 +/− 150 | 125 | 8.5 |
| pH = 8.3 | | | | | |
| oligo non- | 6400 +/− 7200 | 80 +/− 40 | 80 +/− 160 | 182 | 79 |
| functionalized | | | | | |
| pH = 6.8 | | | | | |
| oligo non- | 7500 +/− 9000 | 80 +/− 50 | 75 +/− 70 | 160 | 40 |
| functionalized | | | | | |
| pH = 7.7 | | | | | |
| oligo non- | 7300 +/− 9300 | 50 +/− 15 | 30 +/− 130 | 215 | 74 |
| functionalized | | | | | |
| pH = 8.3 | | | | | |

Legend of Table 3: Experiment consisting of hybridization on a biochip spotted on a surmodics slide: pH test

Oligonucleotide probes functionalized with an amino group (“Oligo NH₂”) and nonfunctionalized oligonucleotide probes (“Oligo nonfunctionalized”) were deposited in solution at various pH values. The biochip was hybridized with a mixture of fluorescence-labeled target oligonucleotides complementary to the probes deposited.

The fluorescence intensity is weak compared to the intensities measured on the slides according to the present invention, all the more so since the scanner settings are very different for the two experiments.

The selectivity of the support with respect to the oligonucleotide probes functionalized with an amino group appears to be good, there is little attachment of the nonfunctionalized oligonucleotide probes.

The grafting appears to be more efficient in basic medium (pH 8.3).

The signal to noise ratio appears to be acceptable, but is much lower than for the slides according to the invention obtained by Method 1 (7 times lower). It is the advantage of having a good fluorescence signal which makes it possible to decrease the intensity of the LASER for excitation and the detection, and thus to decrease the background noise.

Finally, the diameters of the spots are quite large (greater wettability of the “SURMODICS” slides than that of the slides according to the present invention).

A low wettability makes it possible not only to have better definition of the spots but also to reduce the mean distance between two spots, thereby in particular increasing the number of spots of probes deposited per cm².

### Example 7

### Experiments Consisting in Hybridizing the Slides with a Solution of Oligonucleotide Probes: Influence of the Oligonucleotide Concentration

1. Principle of the Experiments

In this experiment, the same sequences as for example 6 are used. In these experiments, the concentration of these sequences is varied during depositing.

2. Material

The same material as that indicated in example 6 is used.

3. Experiment Consisting in Hybridizing Oligonucleotides on the Slides for Aminated Oligonucleotides

a) Preparation of the Slides

The slides are prepared according to Method 1 and are compared with the Surmodics slides. Three oligonucleotide sequences (the same as for example 6) and, for each sequence, two types of modified oligonucleotides (NH₂-functionalized and nonfunctionalized) are deposited. Each oligonucleotide is deposited for 5 concentration values (2, 5, 10, 20 and 40 μM). Each sample is spotted twice. Thus, on each slide, 2 matrices of 2×3×5×2=45 spots of oligonucleotides will have been deposited.

In addition, between each oligonucleotide, a double deposit of buffer is carried out in order to eliminate any risk of contamination.

b) Preparation of the Oligonucleotides

The pH of the solutions to be deposited is fixed at 8.2 by means of 150 mM phosphate buffer.

The oligonucleotide probe concentration is adjusted for the 5 values by making up the volume of the mixture with milliQ water.

c) Preparation of the Hybridization Solution

Same solution and same protocol as for example 6.

d) Hybridization

Same protocol as for example 6.

4. Results of the Experiments

4.1 Slides for Oligonucleotide Probes Functionalized with an Amino Group: Method 1

The scanner is set at 90/95.

The results of the experiments are given in Table 4 below.

TABLE 4

| | | Background | | | Standard |
|---|---|---|---|---|---|
| | Mean | noise | | | deviation |
| | fluorescence | around the | Signal/noise | Mean diameter | of the |
| | intensity | spot | ratio | (μm) | diameters |
| oligo NH₂ | 18000 +/− 1700 | 26 +/− 5 | 700 +/− 200 | 126 | 13 |
| at 40 μM | | | | | |
| oligo NH₂ | 24000 +/− 5000 | 25 +/− 6 | 1000 +/− 350 | 116 | 5 |
| at 20 μM | | | | | |
| oligo NH₂ | 30000 +/− 1500 | 36 +/− 14 | 900 +/− 250 | 112 | 4 |
| at 10 μM | | | | | |
| oligo NH₂ | 31000 +/− 3000 | 24 +/− 3 | 1250 +/− 200 | 112 | 4 |
| at 5 μM | | | | | |
| oligo NH₂ | 12000 +/− 3500 | 25 +/− 4 | 500 +/− 200 | 111 | 5 |
| at 2 μM | | | | | |
| oligo | 7500 +/− 2000 | 27 +/− 3 | 300 +/− 90 | 123 | 4 |
| at 40 μM | | | | | |
| oligo | 10000 +/− 3000 | 26 +/− 3 | 400 +/− 90 | 119 | 5 |
| at 20 μM | | | | | |
| oligo | 14000 +/− 4000 | 24 +/− 5 | 600 +/− 300 | 115 | 5 |
| at 10 μM | | | | | |
| oligo | 13500 +/− 5000 | 30 +/ 4 | 450 +/− 200 | 110 | 0 |
| at 5 μM | | | | | |
| oligo | 11000 +/− 2500 | 28 +/− 7 | 400 +/− 100 | 112 | 4 |
| at 2 μM | | | | | |

Legend of Table 4: Experiment consisting of hybridization on a biochip prepared according to Method 1: concentration test.

NH₂-Functionalized (“oligo NH₂”) and nonfunctionalized oligonucleotide probes were deposited at various concentrations. The chip was hybridized with a mixture of fluorescence-labeled target oligonucleotides complementary to those deposited. 2 matrices of 45 spots were deposited.

This experiment shows that the optimum concentration for depositing the oligos is between 5 and 10 μM. Very good selectivity of the slides is found at these concentrations, since the nonfunctionalized oligos attached less (ratio of 2 for the intensities).

The signal to noise ratio is very good for these optimum concentrations (1000).

The spots are uniform in size.

4.2 Slides Obtained from the Supplier Surmodics for Oligonucleotide Probes Functionalized with an Amino Group

The scanner is set at 90/95. The results of the experiment are given in Table 5 below.

TABLE 5

| | | Background | | | Standard |
|---|---|---|---|---|---|
| | Mean | noise | | Mean diameter | deviation of |
| | fluorescence | around the | Signal/noise | (μm) | the diameters |
| | intensity | spot | ratio | n = 6 | n = 6 |
| oligo NH₂ | 7200 +/− 2800 | 30 +/− 8 | 265 +/− 100 | 171 | 7 |
| at 40 μM | | | | | |
| oligo NH₂ | 4000 +/− 1500 | 25 +/− 7 | 170 +/− 70 | 158 | 7 |
| at 20 μM | | | | | |
| oligo NH₂ | 2700 +/− 1000 | 22 +/− 3 | 125 +/− 60 | 160 | 8 |
| at 10 μM | | | | | |
| oligo NH₂ | 1800 +/− 800 | 23 +/− 6 | 80 +/− 40 | 155 | 5 |
| at 5 μM | | | | | |
| oligo NH₂ | 1100 +/− 500 | 21 +/− 1 | 50 +/− 20 | 152 | 4 |
| at 2 μM | | | | | |
| oligo | 1000 +/− 250 | 22 +/− 3 | 50 +/− 10 | 160 | 10 |
| at 40 μM | | | | | |
| oligo | 500 +/− 130 | 21 +/− 3 | 25 +/− 8 | 158 | 7 |
| at 20 μM | | | | | |
| oligo | 200 +/− 70 | 26 +/− 7 | 8 +/− 3 | 158 | 9 |
| at 10 μM | | | | | |
| oligo | 100 +/− 25 | 21 +/ 3 | 5 +/− 1 | 157 | 7.5 |
| at 5 μM | | | | | |
| oligo | 50 +/− 10 | 22 +/− 4 | 2.5 +/− 0.5 | 155 | 7.6 |
| at 2 μM | | | | | |

Legend of Table 5: Experiment consisting of hybridization on a biochip spotted on a Surmodics slide: concentration test.

The intensities are lower than for the slides of Method 1.

The best signal to noise ratio is obtained for the highest concentration, and is equal to 265.

Compared with the slides according to the invention, the Surmodics slides exhibit a fluorescence signal that is 4 times greater (31000 versus 7200) and a signal to noise ratio that is 5 times greater, for an oligonucleotide concentration that is 8 times lower (5 μM).

Thus, the slides according to the invention make it possible to use a smaller amount of oligonucleotides than the Surmodics slides for a greater signal intensity.

The size of the spots of the Surmodics slides is greater than for the slides according to the invention, with quite a narrow distribution.

The biochips obtained by the methods of production according to the invention, in particular according to Method 1, prove to be particularly effective compared with the other commercial supports, in particular compared with the supports of “Surmodics”0 type which are a reference in the field.

Finally, the experiments carried out with the slides obtained by the methods according to the present invention, in particular according to Method 1, demonstrate:
very efficient grafting (the hybridization signal is very strong),
a very low background noise,
excellent wetting properties (the spots are homogeneous and uniform),
the use of a smaller amount of oligonucleotide probes than for the Surmodics slides.

1-43. (canceled)

44. A method for producing an activated biochip for the covalent attachment of oligonucleotide probes, said biochip comprising a solid support prefunctionalized with a thiol or amine function, said method comprising a step of covalent attachment, under appropriate conditions, to said functionalized support of a spacer compound NHS-PEG-VS of formula (I): 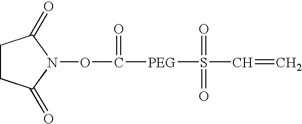
in which PEG denotes poly(ethylene glycol) of formula
HO—(CH₂CH₂O)ₙCH₂CH₂—OH,
where n is an integer chosen such that the molecular mass of the compound NHS-PEG-VS of formula (I) is between 500 and 5000,
said spacer compound being attached to said solid support via a covalent bond resulting either from interaction between the thiol function of said functionalized support and the VS (vinylsulfone) function of the spacer compound of formula (I), or from interaction between the amine function of said functionalized support and the NHS (N-hydroxysuccinimide) function of the spacer compound of formula (I).

45. The method for producing an activated biochip of claim 44, wherein the molecular mass of the compound NHS-PEG-VS of formula (I) is in the region of 3400.

46. The method for producing an activated biochip of claim 44, wherein said solid support is selected from the group consisting of glass, plastic, Nylon®, Kevlar®, silicone, silicon and polysaccharides.

47. The method for producing a biochip of claim 46, wherein said solid support made of glass is functionalized by silanization.

48. The method for producing an activated biochip of claim 44, wherein said solid support is functionalized with an amine function when said oligonucleotide probes intended to be attached are functionalized with an end thiol function.

49. The method for producing an activated biochip of claim 48, wherein said solid support is functionalized in the presence of an aminosilane.

50. The method of claim 49, wherein said aminosilane is N-(2-aminoethyl)-3-aminopropyltrimethoxysilane.

51. The method for producing an activated biochip of claim 44, wherein said solid support is functionalized with a thiol function when said oligonucleotide probes are functionalized with an end amine function.

52. The method for producing an activated biochip of claim 51, wherein said solid support is functionalized in the presence of mercaptosilane.

53. The method of claim 52, wherein said mercaptosilane is (3-mercaptopropyl)trimethoxysilane.

54. The method for producing an activated biochip of claim 44, further comprising a step in which the biochip obtained is frozen, dried or lyophilized.

55. A method for producing a deactivated biochip, for the covalent attachment of oligonucleotide probes functionalized with an end amine function, comprising the following steps:
a) activating the biochip by means of a method for producing an activated biochip of claim 51; and
b) hydrolyzing, in the presence of an aqueous solution, the free NHS functions of the spacer compounds NHS-PEG-VS of formula (I) attached to the solid support.

56. The method for producing a deactivated biochip of claim 55, further comprising a step in which the biochip obtained is frozen, dried or lyophilized.

57. A method for producing a regenerated biochip for the covalent attachment of oligonucleotide probes functionalized with an end amine function, comprising the following steps:
A) deactivating the biochips according to the method of claim 55;
B) regenerating the biochip, in the presence of 1-ethyl-3[3-(dimethylamino)propyl]carbodiimide hydrochloride (EDC) and of NHS, the carboxylate groups obtained at the free end of said spacer compounds after hydrolysis of the free NHS functions initially present.

58. The method of claim 57 further comprising the step of washing the biochip obtained in step B) in deionized water.

59. The method for producing a regenerated biochip of claim 57, further comprising a step in which the biochip obtained is frozen, dried or lyophilized.

60. A method for producing a biochip coated with oligonucleotide probes, comprising:
A) producing an activated biochip by means of the method of claim 44;
B) depositing and attaching, by covalent bonding, under the appropriate conditions, said oligonucleotide probes prefunctionalized with a thiol function, if said solid support has been functionalized with an amine function, or said oligonucleotide probes prefunctionalized with an amine function, if said solid support has been functionalized with a thiol function.

61. The process of claim 60 further comprising removing the oligonucleotide probes which have not attached to the support, by means of rinsing the support under appropriate conditions.

62. The process of claim 61, wherein said rinsing is with deionized water.

63. The method for producing a biochip comprising a solid support prefunctionalized with a thiol function and coated with oligonucleotide probes according to claim 60, wherein said method further comprises deactivating, in the presence of amino compounds under the appropriate conditions, of the NHS functions of the spacer compound which have not interacted with the amine functions of the oligonucleotide probes.

64. The method for producing a biochip of claim 63, wherein said compound for deactivating the NHS functions of the spacer compound is an amino compound having a primary amine.

65. The method for producing a biochip of claim 60, comprising a solid support prefunctionalized with an amine function, and coated with oligonucleotide probes, wherein the method further comprises reducing, under the appropriate conditions, the surface charges in the presence of anionic compounds or compounds capable of establishing covalent bonds with the amine groups and of producing a species that is neutral or negative under the appropriate conditions.

66. The method of claim 65, wherein said reducing occurs in the presence of methyl N-succinimidyl adipate (MSA).

67. The method of claim 60, further comprising a step in which the biochip obtained is conserved in a dry place, in the dark and/or in an inert atmosphere.

68. The method of claim 60, wherein said nucleotide probes are DNAs or RNAs.

69. The method of claim 68, wherein the oligonucleotide probes are single-stranded DNAs or RNAs.

70. The method of claim 68, wherein the size of said oligonucleotide probes is between 15 and 7000 bp.

71. The method of claim 61, wherein the oligonucleotide probes are deposited in the form of spots, the diameters of which are between 20 μm and 500 μm.

72. The method of claim 71, wherein the number of said oligonucleotide probe spots is between 2 and 10⁵.

73. A biochip comprising a solid support prefunctionalized with a thiol or amine function, wherein said biochip comprises a spacer compound NHS-PEG-VS of formula (I): 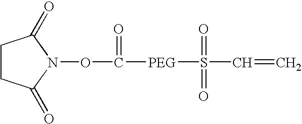
in which PEG denotes poly(ethylene glycol) of formula
HO—(CH₂CH₂O)ₙCH₂CH₂—OH,
where n is an integer chosen such that the molecular mass of the compound NHS-PEG-VS of formula (I) is between 500 and 5000, said spacer compound being attached to said solid support via a covalent bond resulting either from interaction between the thiol function of said functionalized support and the vinylsulfone function of the spacer compound of formula (I), or from interaction between the amine function of said functionalized support and the NHS function of the spacer compound of formula (I).

74. The biochip of claim 73, further comprising at least one oligonucleotide probe, prefunctionalized with a thiol or amine function, attached to said solid support via a covalent bond resulting either from interaction between the free NHS function of said spacer compound of formula (I) and an amine function of said oligonucleotide probe, or from interaction between the free vinylsulfone function of the spacer compound of formula (I) and a thiol function of said oligonucleotide probe.

75. The biochip of claim 74, wherein said attached oligonucleotide probes are single-stranded DNAs or RNAs.

76. The biochip of claim 74, wherein the attached oligonucleotide probes are DNAs or RNAs, the size of which is between 15 and 7000 bp.

77. The biochip of claim 74, wherein the oligonucleotide probes are deposited in the form of spots, the diameters of which are between 20 μm and 500 μm.

78. The biochip of claim 77, wherein the number of oligonucleotide probe spots deposited on the biochip is between 2 and 10⁵.

79. The biochip of claim 73, wherein said solid support is chosen from a solid support selected from the group consisting of glass, plastic, Nylon®, Kevlar®, silicone, and silicon.

80. The biochip of claim 73, wherein the solid support is made of silanized glass.

81. A kit for detecting nucleic acids in a sample, comprising a biochip as claimed in claim 73.

82. A diagnostic instrument or device comprising the biochip of claim 73.

83. A method for detecting nucleic acids in a sample, comprising the following steps:
a) depositing the sample containing the nucleic acids, the detection of whose presence is being sought, on a biochip coated with the oligonucleotide probes of claim 71, under conditions which allow the specific hybridization of these target nucleic acids with said oligonucleotide probes;
b) detecting the nucleic acids captured on the biochip by hybridization.

84. The method of claim 83 wherein the biochip obtained in step a) is rinsed under the appropriate conditions in order to remove the nucleic acids of the sample which have not been captured by hybridization.

85. The method for detecting nucleic acids in a sample of claim 83, wherein the nucleic acids, the detection of whose presence is being sought, are prelabeled at one of their ends with a label capable of directly or indirectly generating a detectable signal.

86. The method of claim 85, wherein the label capable of directly or indirectly generating a detectable signal is a signal detectable by fluorescence.

87. The method of claim 85, wherein at least two of the nucleic acids, the detection of whose presence is being sought, are prelabeled with a different label.

88. The method of claim 85, wherein said label is selected from the group consisting of cyanin derivatives, nanocrystals and nanoparticles.

89. The method of claim 85, wherein said label is a sulfonated derivative of cyanin.

90. The method of claim 85, wherein said label is either Cy5 or Cy3.

91. A method for screening compounds or cells capable of specifically binding to a given oligonucleotide, comprising the following steps:
a) bringing said test compound into contact with the biochip of claim 74, under the conditions for the possible specific binding of said test compound or of said test cell with said given oligonucleotide, said biochip comprising at least one spot of oligonucleotide probes containing said given oligonucleotides attached to its solid support and, wherein said compound or said cell being labeled with a label capable of directly or indirectly generating a detectable signal;
b) removing, by means of at least one washing step under the appropriate conditions, the test compounds or cells not specifically bound to said given oligonucleotide; and
c) selecting the compound or said cell specifically bound to said given oligonucleotide, where appropriate by selecting the compound or said cell whose signal has been detected at the spot containing said given oligonucleotide.
